(19) United States
(12) Patent Application Publication
(10) Pub. No.: US 2010/0100185 A1
(43) Pub. Date: Apr. 22, 2010

(54) Intervertebral Disc Prosthesis Having Viscoelastic Properties

(75) Inventors: Hai Trieu, Cordova, TN (US); Jeff Nycz, Warsaw, IN (US); Jeff Justis, Germantown, TN (US); Dimitri Protopsaltis, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/256,008

(22) Filed: Oct. 22, 2008

(51) Int. Cl. A61F 2/44

(52) U.S. Cl. 623/17.16; 623/17.11

(57) ABSTRACT

A motion-preserving implant device for insertion between two bones is disclosed. The motion-preserving implant includes a first plate member for engaging with a first bone and a second plate member for engaging with a second bone. A viscoelastic component is positioned between the two plate members that is configured to constrain and dampen the relative motion between the two plate members.

### BACKGROUND

The present disclosure relates generally to the field of orthopedics and, in particular, intervertebral prosthetic joints for use in the total or partial replacement of a natural intervertebral disc, and methods for use therewith.

In the treatment of diseases, injuries or malformations to bone joints, such as those affecting spinal motion segments, and especially those affecting disc tissue, it has long been known to remove some or all of a degenerated, ruptured or otherwise failing disc. In cases involving intervertebral disc tissue that has been removed or is otherwise absent from a spinal motion segment, corrective measures are taken to ensure the proper spacing of the vertebrae formerly separated by the removed disc tissue.

In some instances, the two adjacent vertebrae are fused together using transplanted bone tissue, an artificial fusion component, or other compositions or devices. Spinal fusion procedures, however, have raised concerns in the medical community that the biomechanical rigidity of intervertebral fusion may predispose neighboring spinal motion segments to rapid deterioration. More specifically, unlike a natural intervertebral disc, spinal fusion prevents the fused vertebrae from pivoting and rotating with respect to one another. Such lack of mobility tends to increase stresses on adjacent spinal motion segments. Additionally, several conditions may develop within adjacent spinal motion segments, including disc degeneration, disc herniation, instability, spinal stenosis, spondylosis and facet joint arthritis. Consequently, many patients may require additional disc removal and/or another type of surgical procedure as a result of spinal fusion. Alternatives to spinal fusion are therefore desirable.

In other instances, intervertebral disc arthroplasty devices have been proposed for preventing the collapse of the intervertebral space between adjacent vertebrae while maintaining a certain range of pivotal and/or rotational motion therebetween. Such devices typically include articular elements positioned between upper and lower plates, which are further attached to respective superior and inferior vertebrae. The articular elements are anchored to the upper and lower vertebrae by a number of methods, including the use of bone screws that pass through corresponding openings in each of the elements and thread into vertebral bone, and/or by the inclusion of spikes or teeth that penetrate the vertebral endplates to inhibit migration or expulsion of the device. The articular elements are typically configured to allow the vertebrae to pivot and/or rotate relative to one another. These motion-preserving devices, however, do not precisely simulate the natural biomechanics of a native disc, and there is a general need in the industry to provide an improved intervertebral prosthetic joint. The present invention addresses this and other needs in a novel and non-obvious manner.

### SUMMARY

The present application relates generally to intervertebral prosthetic devices and methods for making and using same. While the actual nature of the invention covered herein can only be determined with reference to the claims appended hereto, certain forms of the invention that are characteristic of the preferred embodiments disclosed herein are described briefly as follows.

In one form, a motion-preserving implant device includes a first plate member for engaging with a first vertebral body; a second plate member for engaging with a second vertebral body; and a viscoelastic component between the two plate members. The viscoelastic component defines at least two fluid chambers having elastic side walls and at least one channel fluidly connecting the at least two chambers. The device also includes a fluid contained within said chambers. In another embodiment, the device includes at least three fluid chambers, each of the three fluid chambers fluidly connected to at least one other fluid chamber. In yet another embodiment, the device includes at least four fluid chambers, each of the four fluid chambers fluidly connected to at least one other fluid chamber. In yet another embodiment, the device includes at least five fluid chambers, each of the five fluid chambers fluidly connected to at least one other fluid chamber.

In another form, an intervertebral prosthetic device includes a first component adapted to engage a first vertebra and including a first articular surface; a second component adapted to engage a second vertebra and including a second articular surface that cooperates with said first articular surface at an articulating interface to permit articulating motion between said first and second components; and a viscoelastic component positioned about said articulating interface, said viscoelastic component defining at least two fluid chambers having elastic side walls and at least one channel fluidly connecting said at least two chambers. The device also includes a fluid contained within said chambers. In another embodiment, the device includes at least three fluid chambers, each of the three fluid chambers fluidly connected to at least one other fluid chamber. In yet another embodiment, the device includes at least four fluid chambers, each of the four fluid chambers fluidly connected to at least one other fluid chamber. In yet another embodiment, the device includes at least five fluid chambers, each of the five fluid chambers fluidly connected to at least one other fluid chamber.

In yet another form, an intervertebral prosthetic joint system includes two implant devices configured for bilateral placement in an interbody disc space. Each of the devices includes a first plate member for engaging with a first vertebral body; a second plate member for engaging with a second vertebral body; and a viscoelastic component between the two plate members. The viscoelastic component of each device includes at least two fluid chambers having at least one channel fluidly connecting the at least two chambers. Each of the devices also includes a fluid contained within the chambers. In another embodiment, at least one of the devices includes at least three fluid chambers, each of the three fluid chambers fluidly connected to at least one other fluid chamber. In yet another embodiment, at least one of the devices includes at least four fluid chambers, each of the four fluid chambers fluidly connected to at least one other fluid chamber. In yet another embodiment, at least one of the devices includes at least five fluid chambers, each of the five fluid chambers fluidly connected to at least one other fluid chamber.

In still another form, an intervertebral prosthetic joint system includes at least three implant devices configured for placement in an interbody disc space. At least two of the devices includes a first plate member for engaging with a first vertebral body; a second plate member for engaging with a second vertebral body; and a viscoelastic component between the two plate members. In one embodiment, all of the at least three implant devices includes a first plate member for engaging with a first vertebral body; a second plate member for engaging with a second vertebral body; and a viscoelastic component between the two plate members. In another embodiment, at least one of the devices does not include a viscoelastic component. The viscoelastic component includes at least two fluid chambers having at least one channel fluidly connecting the at least two chambers.

In still yet another form, an intervertebral prosthetic device includes a first component including means for engaging a first vertebra; a second component including means for engaging a second vertebra; and means positioned between the first and second components for imparting viscoelastic movement between the first and second components.

These and other aspects of the invention will be discussed further below.

### BRIEF DESCRIPTION OF THE DRAWINGS

FIG. 1 is a lateral view of a portion of the spinal column, illustrating a pair of adjacent upper and lower vertebrae separated by a natural intervertebral disc.

FIG. 2 is a perspective view of one embodiment intervertebral prosthetic implant.

FIG. 3 is a cutaway plan view of the embodiment depicted in FIG. 2.

FIG. 4 is an elevation cross section view of the embodiment depicted in FIG. 2, taken along line 4-4 shown in FIG. 3.

FIG. 5 is a cutaway plan view of another embodiment intervertebral prosthetic implant.

FIG. 6 is an elevation cross section view of another embodiment intervertebral prosthetic implant.

FIGS. 7A and 7B are cross section views of a fluid chamber containing a magnetic rheological (MR) fluid.

FIG. 8 is an elevation cross section view of another embodiment intervertebral prosthetic implant.

FIG. 9 is an elevation cross section view of another embodiment intervertebral prosthetic implant.

FIG. 10 is an exploded perspective view of another embodiment intervertebral prosthetic implant.

FIG. 11 is an elevation cross section view of the embodiment depicted in FIG. 10.

FIG. 12 is an exploded perspective view of another embodiment intervertebral prosthetic implant.

FIG. 13 an anterior view of the portion of the spinal column shown in FIG. 1, illustrating the removal of portions of the upper and lower vertebrae to accommodate insertion of an intervertebral prosthetic joint therebetween.

FIG. 14 is a lateral view of the portion of the spinal column shown in FIG. 13.

FIG. 15 is an anterior view of the portion of the spinal column shown in FIG. 13, illustrating implantation of the intervertebral prosthetic joint between the upper and lower vertebrae.

FIG. 16 is a partial sectional view of the spinal column portion shown in FIG. 13, illustrating implantation of the intervertebral prosthetic joint between the upper and lower vertebrae.

FIG. 17 is a cutaway plan view of a bilateral embodiment intervertebral prosthetic implant system.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

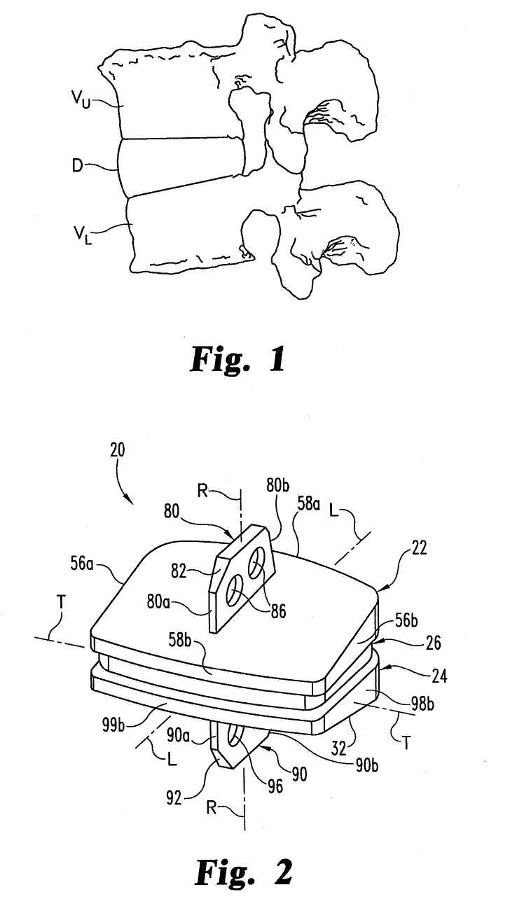

Referring to FIG. 1, shown therein is a lateral view of a portion of a human spinal column, illustrating a pair of adjacent upper and lower vertebrae V_{U}, V_{L} separated by a natural intervertebral disc D, together with form a joint, or motion segment, in which one or more embodiments of the present application can be implemented. As discussed above, in cases where the natural intervertebral disc D is diseased, injured or degenerated (collectively, “damaged”), the natural disc D is typically removed via a discectomy or a similar surgical procedure, the details of which would be known to one of ordinary skill in the art. Some or all of the damaged disc 12 may be replaced by a motion-preserving intervertebral disc prosthesis according to one or more embodiments of the present application. Although spinal products are discussed in detail, other embodiments are anticipated, including those related to large-scale orthopedics such as hips and knees, small scale orthopedics such as fingers and wrists, and dental-related products.

Depicted in FIG. 2 is an embodiment motion-preserving implant device 20 (also referred to herein as “intervertebral prosthetic joint 20” or “prosthetic joint 20” or “intervertebral prosthetic device 20” or articulating device 20”) that can be placed between vertebrae V_{U} and V_{L}. Device 20 includes a first plate member 22 for engaging with the first (e.g., upper, or superior) vertebrae V_{U} and a second plate member 24 for engaging with the second (e.g., lower, or inferior) vertebrae V_{L}. Device 20 further includes an intermediate viscoelastic component 26 positioned between first plate member 22 and second plate member 24 for providing a limited degree of lateral, transverse and rotational movement between first and second plate members 22, 24. Viscoelastic component 26 defines at least two chambers that are fluidly connected to one another through a channel that is operable to allow a fluid to flow from between the chambers in response to fluctuating compressive loads applied to the respective chambers. In another embodiment, viscoelastic component 26 includes at least three fluid chambers, each of the three fluid chambers fluidly connected to at least one other fluid chamber. In yet another embodiment, the device includes at least four fluid chambers, each of the four fluid chambers fluidly connected to at least one other fluid chamber. In yet another embodiment, the device includes at least five fluid chambers, each of the five fluid chambers fluidly connected to at least one other fluid chamber. Plate members 22, 24 and viscoelastic component 26 cooperate to form the prosthetic joint which is sized and configured for disposition within the intervertebral space between adjacent vertebral bodies V_{U}, V_{L}.

Articulating joint 20 provides relative pivotal and rotational movement between the adjacent vertebral bodies to maintain or restore motion that mimics the normal biomechanical motion provided by a natural intervertebral disc. More specifically, plate members 22, 24 are permitted to pivot relative to one another about a number of axes, including lateral or side-to-side pivotal movement about longitudinal axis L and anterior-posterior pivotal movement about a transverse axis T. It should be understood that in a preferred embodiment of the invention, plate members 22, 24 are permitted to pivot relative to one another about any axes that lies in a plane that intersects longitudinal axis L and transverse axis T. Additionally, plate members 22, 24 are preferably permitted to rotate relative to one another about a rotational axis R. Although the prosthetic joint 20 has been illustrated and described as providing a specific combination of motions, it should be understood that other types and combinations of movement are also possible and are contemplated as falling within the scope of the present application, such as, for example, relative translational or linear motion.

Although plate members 22, 24 of prosthetic joint 20 may be formed from a wide variety of materials, in one embodiment, plate members 22, 24 are formed of a cobalt-chrome-molybdenum metallic alloy (ASTM F799 or F-75). Also, at least a portion of the plates can be coated with an amorphous oxide coating. In alternative embodiments, plate members 22, 24 may be formed of other metallic materials such as titanium or stainless steel, a ceramic, a polymeric material such as polyethylene or PEEK, or any other biocompatible material that would be apparent to one of ordinary skill in the art. The surfaces of plate members 22, 24 that are positioned in direct contact with vertebral bone can be coated with a bone-growth promoting substance, such as, for example, a hydroxyapatite coating formed of calcium phosphate. Additionally, the surface of plate members 22, 24 that are positioned in direct contact with vertebral bone optionally can be roughened prior to being coated with the bone-growth promoting substance to further enhance bone on-growth. Such surface roughening may be accomplished by way of, for example, acid etching, knurling, application of a bead coating, or other methods of roughening that would occur to one of ordinary skill in the art.

Upper plate member 22 has a lower surface 52 and an opposite bearing surface 54. Bearing surface 54 is preferably sized and shaped to substantially correspond to the size and shape of the vertebral endplate of an adjacent vertebra. Lower surface 52 and bearing surface 54 are separated by a pair of laterally facing surfaces 56a, 56b and a pair of axially facing surfaces 58a, 58b. The laterally facing surfaces 56a, 56b can optionally define channels (not shown) extending along at least a portion of the length of the plate member 22, which channels can be configured to engage a corresponding portion of a surgical instrument (not shown) to aid in the manipulation and insertion of the prosthetic joint 20 within an intervertebral space between adjacent vertebrae. The surgical instrument (not shown) is preferably configured to hold plate members 22, 24 at a predetermined orientation and spatial relationship relative to one another during manipulation and insertion of prosthetic joint 20, and to release plate members 22, 24 once properly positioned between the adjacent vertebrae.

There are a variety of ways in which plates 22, 24 can be attached to vertebrae V_{U}, V_{L}, including but not limited to using a flange member or keel, a lip portion that extends around the vertebral body for receiving one or more bone screws, and other configurations known in the art. In the embodiment depicted in FIGS. 2-4, flange member or keel 80 extends from bearing surface 54 and is configured for disposition within a preformed opening in the adjacent vertebral endplate. In one embodiment, keel 80 extends perpendicularly from bearing surface 54 and is approximately centrally located along bearing surface 54. However, it should be understood that other positions and orientations of keel 80 are also contemplated. It should also be understood that plate member 22 may include two or more keels 80 extending from bearing surface 54.

Keel 80 extends from a location adjacent the axially facing surface 58a toward the axially facing surface 58b along a substantial portion of plate member 22. In one embodiment, keel 80 extends along substantially the entire length of plate member 22. In one embodiment (not shown) keel 80 is wedge-shaped, defining an outward taper as the keel 80 extends from a leading or insertion end 80a towards a trailing end 80b. As will become apparent, an outward taper aids in the insertion of keel 80 within preformed openings in the adjacent vertebrae. In one specific embodiment, the outward taper is about 4 degrees. However, other taper angles are also contemplated. It should also be understood that keel 80 need not necessarily be tapered along its length. Additionally, insertion end 80a of keel 80 optionally can include a beveled surface 82 to further aid in the implantation of prosthetic joint 20.

In another embodiment, keel 80 may alternatively extend between the laterally facing surface 56a, 56b along a substantial portion of plate member 22. As described further hereinbelow, such an embodiment would accommodate insertion of prosthetic joint 20 using a lateral approach as opposed to the anterior approach illustrated in FIGS. 14-17. In a further embodiment, keel 80 may be tapered along its height, either tapering inwardly from bearing surface 54 to define a wedge shape or tapering outwardly from bearing surface 54 to define a dove-tail shape. In still another embodiment, keel 80 may be configured as a winged keel, including a transverse portion extending across the main body portion of keel 80.

Keel 80 also includes a pair of openings 86 extending therethrough to facilitate bone through-growth to enhance fixation to the adjacent vertebra. However, it should be understood that any number of openings 86 may be defined through keel 80, including a single opening or three or more openings. It should also be understood that openings 86 need not necessarily extend entirely through keel 80, but may alternatively extend partially therethrough. It should further be understood that keel 80 need not necessarily define any openings 86 extending either partially or entirely therethrough. Additionally, although openings 86 are illustrated as having a circular configuration, it should be understood that other sizes and configures of openings 86 are also contemplated. As discussed above, the surfaces of plate member 22 that are in direct contact with vertebral bone can be coated with a bone-growth promoting substance. Specifically, bearing surface 54 and bone-facing surfaces of keel 80 can be coated with hydroxyapatite or other bone-growth promoting substance to promote bony engagement with the adjacent vertebrae. As also discussed above, bearing surface 54 and bone-facing surfaces of keel 80 can be roughened prior to application of the hydroxyapatite coating.

Lower plate member 24 has an upper surface 102 and an opposite bearing surface 104. Bearing surface 104 is preferably sized and shaped to substantially correspond to the size and shape of the vertebral endplate of an adjacent vertebra. Upper surface 102 and bearing surface 104 are separated by a pair of laterally facing surfaces 98a, 98b and a pair of axially facing surfaces 99a, 99b. Laterally facing surfaces can optionally define channels (not shown) extending along at least a portion of the length of plate member 24. The channels (not shown), if present, are configured to engage a corresponding portion of a surgical instrument (not shown) to aid in the manipulation and insertion of prosthetic joint 20.

A flange member or keel 90, configured similar to keel 80 of plate member 22, extends from bearing surface 104. In one embodiment, keel 90 extends perpendicularly from bearing surface 104 and is approximately centrally located along bearing surface 104. However, it should be understood that other positions and orientations of keel 90 are also contemplated. It should also be understood that plate member 24 may include two or more keels 90 extending from bearing surface 104.

Keel 90 extends from a location adjacent axially facing surface 99a toward axially facing surface 99b, preferably along a substantial portion of plate member 24. As with keel 80, keel 90 can be wedge-shaped, defining an outward taper as it extends from a leading or insertion end 90a to trailing end 90b. Additionally, insertion end 90a of keel 90 includes a beveled surface 92 to further aid in the implantation of the prosthetic joint 20. In another embodiment, keel 90 may alternatively extend between the laterally facing surfaces 98a, 98b along a substantial portion of bearing surface 104 to accommodate insertion of the prosthetic joint 20 between adjacent vertebral bodies using a lateral approach. In a further embodiment, keel 90 may be tapered along its height, either tapering inwardly from bearing surface 104 to define a wedge shape or tapering outwardly from bearing surface 104 to define a dove-tail shape. In still another embodiment, keel 90 may be configured as a winged keel, including a transverse portion extending across the main body portion of keel 90.

As with keel 80, keel 90 includes a pair of openings 96 extending therethrough to facilitate bone through-growth to enhance fixation to the adjacent vertebra. However, it should be understood that any number of openings 96 may be defined through keel 90, including a single opening or three or more openings. It should also be understood that the openings 96 need not necessarily extend entirely through keel 90, but may alternatively extend partially therethrough. It should further be understood that keel 90 need not necessarily define any openings 96 extending either partially or entirely therethrough. As discussed above, the surfaces of plate member 24 that are in direct contact with vertebral bone can be coated with a bone-growth promoting substance, such as, for example, a hydroxyapatite coating or a coating of another bone-growth promoting substance. As also discussed above, the surfaces of plate member 24 that are in direct contact with vertebral bone can be roughened prior to application of the bone-growth promoting substance.

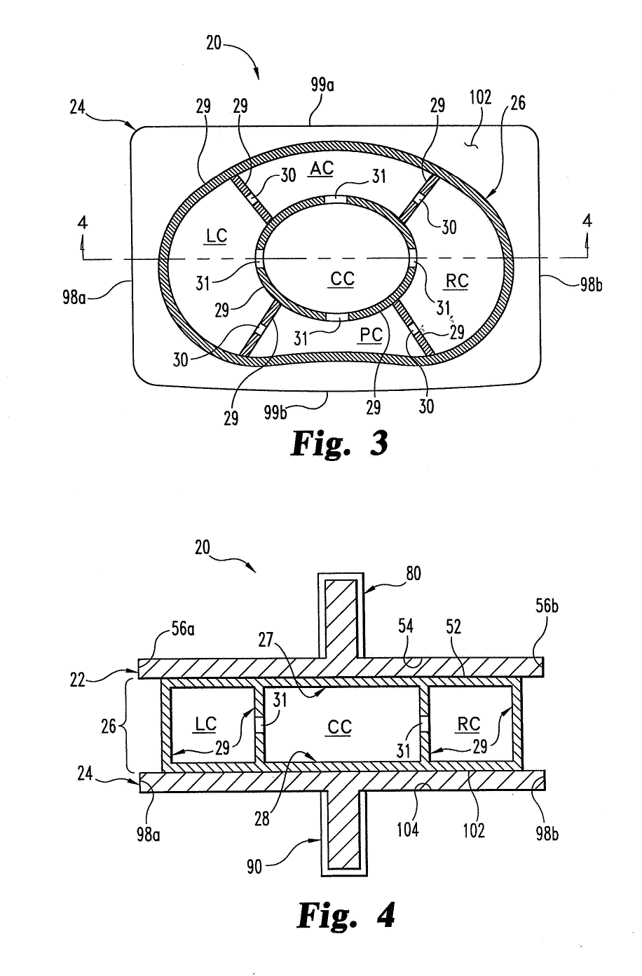

Viscoelastic component 26 is positioned between plate member 22 and plate member 24, and is operable to allow limited pivotal and rotational movement between plate members 22, 24, thereby also allowing limited pivotal and rotational movement between vertebrae V_{U} and V_{L} after implantation and ingrowth of bone, by which plate member 22 is rigidly affixed to vertebra V_{U} and plate member is rigidly affixed to vertebra V_{L}. With reference to FIGS. 3 and 4, viscoelastic component 26 includes a first wall 27 adjacent plate member 22, a second wall 28 adjacent plate member 24 and walls 29 generally perpendicular to walls 27 and 28 that operate together with walls 27 and 28 to define five chambers within viscoelastic component 26. The five chambers are identified in FIGS. 3 and 4 as a central chamber CC, an anterior chamber AC, a posterior chamber PC and two lateral chambers identified as left chamber LC and a right chamber RC. Each of the five chambers is connected to adjacent chambers through a channel or aperture, identified as channels 30 and channels 31. Walls 27, 28, 29 have sufficient thickness, and are made of a suitable material, to impart long-term durability and performance to device 20. Examples of suitable materials for use in making walls 27, 28, 29 include, without limitation, silicone, polyurethane, silicone-polyurethane or polyolefin rubber. In addition, walls 27, 28, 29 can include reinforcing structures if it is desired to impart additional rigidity to the walls. Before, during or after implantation of device 20 into a patient an intervertebral space in a patient, a biocompatible fluid is introduced into chambers CC, AC, PC, LC and RC. Examples of fluids that can be used include, without limitation, water, saline, polyethylene glycol, glycerol, plasma extender, hydrocarbon solvents, polymer solutions, polymeric gels, hydrogels, hydrogel solutions and the like. In one embodiment, the fluid is one that has a viscosity of from about 1 to about 250,000 Centipoise. In another embodiment, the fluid has a viscosity of from about 100 to about 50,000 Centipoise.

In use, viscoelastic component 26 is operable to deform upon application of pivotal or rotational stresses to walls 27, 28 by plate members 22, 24, respectively. Deformation occurs by flexing and/or bending of walls 29 and by flow of the fluid from one chamber of component 26 to one or more other chambers. For example, a force urging laterally facing surface 56a of plate member 22 toward laterally facing surface 98a of plate member 24 will exert a compressive force on chamber LC, causing walls 29 defining chamber LC to flex and/or bend, and creating a pressure differential between chamber LC and adjacent chambers AC, CC, PC. As a result of the pressure differential, fluid residing in chamber LC will pass into chamber AC through the aperture 30 in the wall 29 separating chamber LC from chamber AC, into chamber CC through the aperture 31 in the wall 29 separating chamber LC from chamber CC, and/or into chamber PC through the aperture 30 in the wall 29 separating chamber LC from chamber PC. The compressive force thereby results in laterally facing surfaces 56a, 98a moving toward one another.

Apertures 30, 31 can be sized and configured to control the rate of fluid flow from one chamber to another. In one embodiment, apertures 30 in the walls 29 separating chambers LC, AC, RC and PC from one another are more flow restrictive than apertures 31 in the walls 29 separating chambers LC, AC, RC and PC from chamber CC. This can be achieved by making apertures 30 smaller than apertures 31, by controlling the flexibility of the material surrounding apertures 30 or by other means. In another embodiment, the apertures 30 in the walls 29 separating chambers LC, AC, RC and PC from one another are absent, and all fluid flow between chambers LC, AC, RC and PC is routed through central chamber CC via apertures 31. In yet another embodiment, apertures 31 comprise valves that control flow between the respective chambers. For example, in one embodiment, the apertures 31 in the walls 29 separating chambers LC, AC, RC and PC from chamber CC are configured to restrict flow of fluid from chambers LC, AC, RC, PC into central chamber CC when a compressive force urges fluid from one or more of chambers LC, AC, RC, PC into central chamber CC, but to allow free flow of fluid in the reverse direction, i.e., from central chamber CC into chambers LC, AC, RC, PC. For example, in embodiments in which apertures 30 are present, the restriction of flow from chambers LC, AC, RC, PC into central chamber CC by valves present at apertures 31 can be designed to match the level of flow restriction imparted by apertures 30, thereby controlling the rate of flow from chambers LC, AC, RC, PC and dampening the relative movement of plates 22 and 24 when a significant compressive force is placed on any one of chambers LC, AC, RC, PC. In contrast, the free flow of fluid from central chamber CC into chambers LC, AC, RC, PC allowed by the valve will allow fluid to more easily pass back into chambers LC, AC, RC, PC, thereby allowing plates 22 and 24 to more easily return to their original (generally parallel) position, when the compressive force is removed. This effect is even more pronounced in embodiments in which apertures 30 are absent. In still other embodiments, one or more of apertures 30 can also comprise a valve to provide increased control over fluid flow.

Viscoelastic component 26 can be adhered or otherwise affixed to plates 22, 24. In other embodiments, viscoelastic component is not adhered or otherwise affixed to plates 22, 24. In such embodiments, other structures (not shown) are present to maintain plates 22, 24 in an appropriate orientation. In this way, viscoelastic component 26 is in continual contact with the plates 22, 24 but are only semi-constrained, allowing some rotational “slide” between plates 22, 24 and viscoelastic component 26. It is understood that, in addition to sliding movement, additional movement may still be provided due to the elastic nature of viscoelastic component 26.

In another embodiment, depicted in FIG. 5, device 120 includes a viscoelastic component 126 structured as described above in connection with viscoelastic component 26; however, viscoelastic component 126 also includes a more robust load bearing chamber LBC within central chamber CC. Chamber LBC is defined by a thicker, more durable wall 132 than walls 29, and has more load bearing capacity than walls 29 and chambers AC, LC, RC, PC, CC, but is sufficiently flexible to allow relative pivotal, rotational and compressive motion between plate components 22, 24. In this embodiment, each of the viscoelastic component-facing surfaces of plates 122, 124 can optionally define a recess (not shown), and opposite portions of the walls of chamber LBC can reside within the recesses.

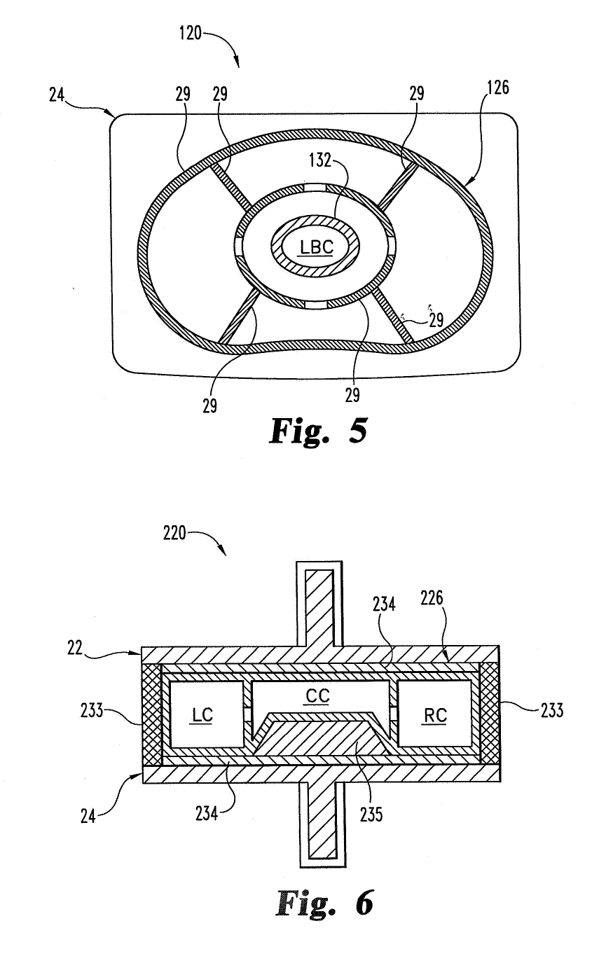

Also, as shown in U.S. Patent Publication No. 2002/0035400, which is incorporated herein by reference, a sheath can be used to enclose some or all of the area between the two plates 22, 24, including viscoelastic component 26. In another embodiment, the viscoelastic component can include structures in addition to, or in place of, the load bearing chamber LBC discussed above to provide additional mechanical support to the intervertebral prosthetic device. For example, intervertebral prosthetic device 220 depicted in FIG. 6 includes a peripheral band 233 positioned between outer surfaces of plate members 22, 24, which can operate as a sheath in addition to providing mechanical support. Peripheral band 233 can extend entirely around viscoelastic component 26 or can extend about only portions of viscoelastic component 26. Peripheral band 233 can be structured, for example, by selection of dimensions and materials, to have a wide variety of flexibilities, and thus to provide varying degrees of mechanical support. In another embodiment, device 220 can include vertical posts or other bumpers extending between plate members 22, 24, either at peripheral locations or more central locations relative to plate members 22, 24.

In yet another embodiment, some or all of viscoelastic component 26 and/or the fluid contained in the fluid chambers can be made of a material that changes properties in response to an external stimulus, such as a radio-frequency signal. For example, device 20 may require additional cushioning or constraint during a period in which a spondylolisthesis condition is first addressed, but as the spondylolisthesis resides, the cushioning or constraint can be reduced or removed. In one embodiment of the present application, the fluid positioned in chambers AC, LC, RC, PC, CC and/or LBC includes magnetically sensitive particles entrained therein, which can be utilized to modify the rheological behavior of viscoelastic component 26. Such a fluid is referred to herein as a “magnetic rheological” or “magnetorheological” (MR) fluid. In an external magnetic field, MR fluids change to a semi-solid state. In one embodiment, the MR fluid is a suspension of micron sized iron particles in a viscous medium. In another embodiment, the magnetically sensitive particles include a polymer coating thereon. Polymer coating of particles can be achieved, for example, through a process called Atom Transfer Radical Polymerization (ATRP). One embodiment MR fluid includes ferromagnetic particles dispersed in a carrier medium of PAO, with polyurethane as a stabilizer. Another embodiment MR fluid includes carbonyl iron particles surface grafted with butyl acrylate, in a carrier medium of N-octyl-pyrrolidone. Yet another embodiment MR fluid includes carbonyl iron particles surface grafted with pentafluorostyrene, in a carrier medium of N-octyl-pyrrolidone. The MR fluid exhibits controllable and reversible changes in its rheological properties under an externally applied magnetic field. Moreover, the properties of the MR fluid, and its response characteristics to externally applied conditions, can be varied dependent upon the nature, size and density of the particles, fluid structure, carrier fluids, additives and applied magnetic field, among other factors. The MR fluid can also be formulated to improve biocompatibility for use in the present application, if desired.

When a MR fluid is used, intervertebral prosthetic device 20, 120, 220 can also include a magnetic field source to have an affect on the fluid in the chambers of the viscoelastic component. With reference to FIG. 6, magnetic field source 234 is affixed to plate member 24 at one or more locations suitable for applying a magnetic field across one or more of the fluid chambers of viscoelastic component 226. In alternative embodiments, source 234 can be a permanent magnet or an electromagnet. In one embodiment in which source 234 is an electromagnet, the electromagnet can be adjusted to create magnetic fields of varying strength, thereby providing for the adjustment of the rheological behavior of the fluid in central chamber CC (and optionally also in chambers LC, RC, AC, PC), which will, in turn, modify the biomechanical behavior of intervertebral prosthetic device 20, 120, 220. This can be advantageous, for example, where differing levels of stiffness, motion and/or responsiveness are desirable to suit a given patient and/or a given set of circumstances in which intervertebral prosthetic device 20, 120, 220 operates. Viscoelastic component 226 also includes a compartment 235 that houses electronic components, which can include, for example, a microelectronic controller, a battery or other power source, an antenna, an accelerometer and/or a sensor. In various alternative embodiments, the microelectronic controller can operate to receive inputs from a remote device, transmit data to a remote device, such as, for example, data collected by sensors, adjust the strength of the magnetic field and the like.

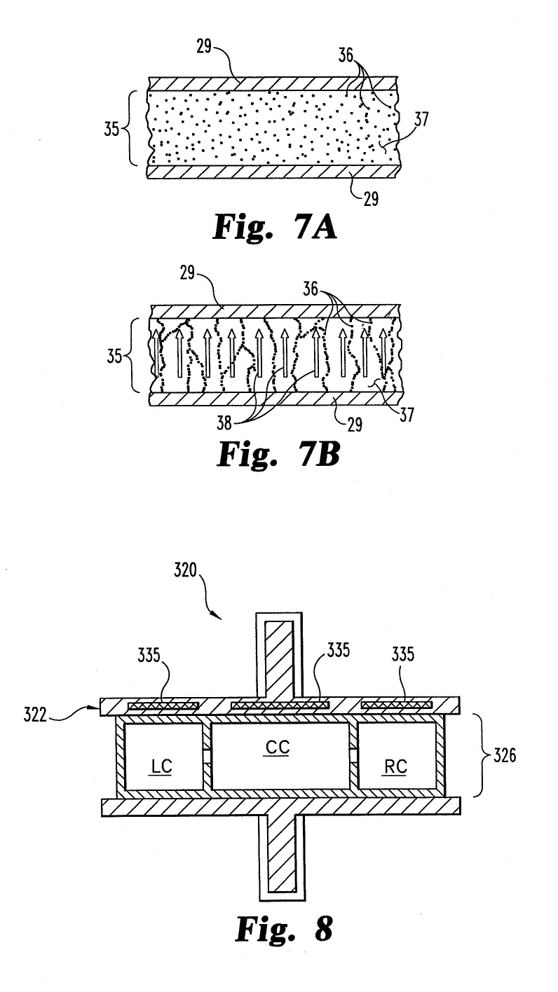

FIGS. 7A and 7B schematically represent MR fluid 35 within central chamber CC in the absence of a magnetic field (FIG. 7A) and in the presence of a magnetic field (FIG. 7B). MR fluid 35 in this embodiment includes a plurality of magnetically sensitive particles 36 entrained in fluid 37. Particles 36 can be, for example, iron particles and/or iron particles grafted with a polymeric material such as, for example, butyl acrylate, and fluid 37 can be, for example, N-butyl pyrrolidone. In FIG. 7B, the direction of magnetic flux is represented by arrows 38. In the absence of a magnetic field across chamber CC (FIG. 7A), magnetic particles 36 are shown randomly dispersed in fluid 37. It is also possible that particles 36 might settle to some extent when intervertebral prosthetic device 20, 120, 220 is at rest. When a magnetic field is applied across chamber CC, however, magnetic particles 36 are believed to form chains in the direction of flux lines 38. The formation of these chains increases the resistance of intervertebral prosthetic device 20, 120, 220 to relative motion between plate member 22 and plate member 24 when the magnetic field is applied, and the resistance can be adjusted by adjusting the strength of the magnetic field. While the present application is not intended to be limited by any theory or mechanism by which it achieves a given result, it is believed that the increased resistance to motion results from the chains of magnetic particles providing mechanical support to plate members 22, 24 and/or from the chains of magnetic particles operating to modulate fluid flow between the respective chambers of viscoelastic component 26, 126, 226, 326. By applying magnetic fields of varying intensities, the mechanical properties of the prosthetic device can be varied for different applications and/or patient needs (e.g., faster responding versus slower responding behaviors). In embodiments that include electromagnets, properties can be adjusted even following implantation by transmitting a wireless signal from an external device to a receiver in compartment 235, which operates to adjust the strength of the magnetic field generated by the electromagnet.

In another embodiment, depicted in FIG. 8, intervertebral prosthetic device 320 includes a plurality of magnetic field sources 334, each one corresponding to one of the fluid chambers. As with source 234, each of sources 334 can be can be a permanent magnet or an electromagnet. In one embodiment in which one or more of sources 334 is an electromagnet, the electromagnet can be adjusted to create magnetic fields of varying strength, thereby providing for the adjustment of the rheological behavior of the fluid in the corresponding fluid chambers, which will, in turn, modify the biomechanical behavior of intervertebral prosthetic device 320. In device 320, magnetic field sources 334 are positioned within plate member 322; however, a person skilled in the art will readily appreciate that sources 334 can optionally be positioned between plate member 324 and viscoelastic component 326, in slots opening through plate member 324, or in a variety of other locations suitable for applying desired magnetic fields across one or more of the fluid chambers.

In another embodiment, depicted in FIG. 9, intervertebral prosthetic device 420 has a configuration similar to device 220 depicted in FIG. 6, and also includes a bumper 436 positioned between plate member 22 and central chamber CC. Bumper 436 is positioned opposite compartment 435 and defines a space between bumper 436 and compartment 435. Bumper operates to limit the amount of axial compression of device 420 to the width of the space between bumper 436 and compartment 435. Bumper 436 can be made of a wide variety of materials including, for example, solid polymer materials or metals. As will be appreciated by a person skilled in the art, while one bumper embodiment is depicted in FIG. 9, intervertebral prosthetic devices in accordance with the present application can alternatively include bumpers having a wide variety of configurations and positioned in a wide variety of locations in the device, provided that the bumper defines a space between one or both of the plate members, or between bumpers affixed thereto, to limit the amount of axial compression of the device.

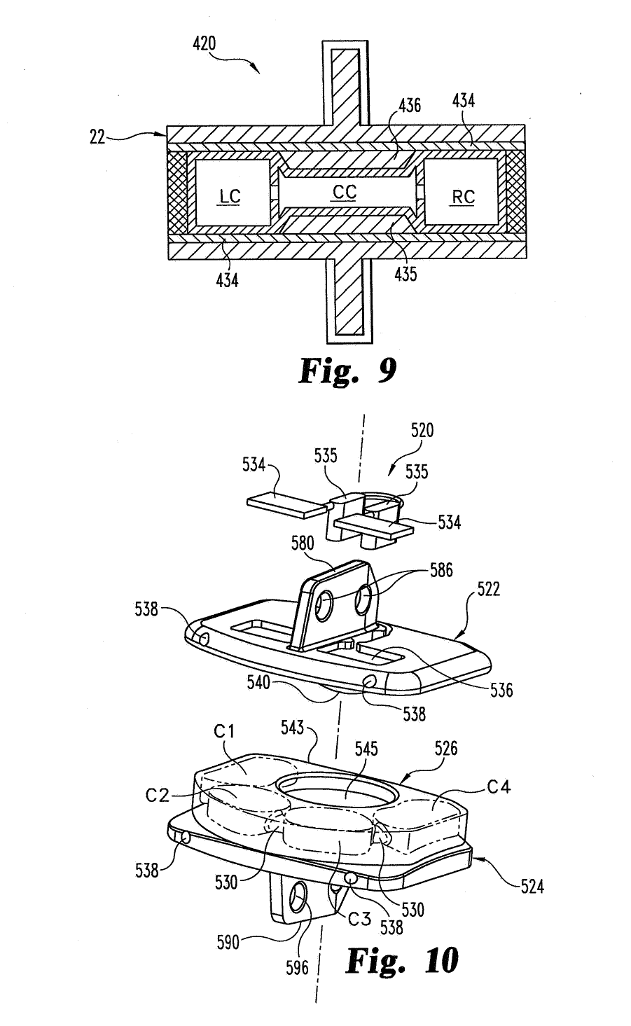

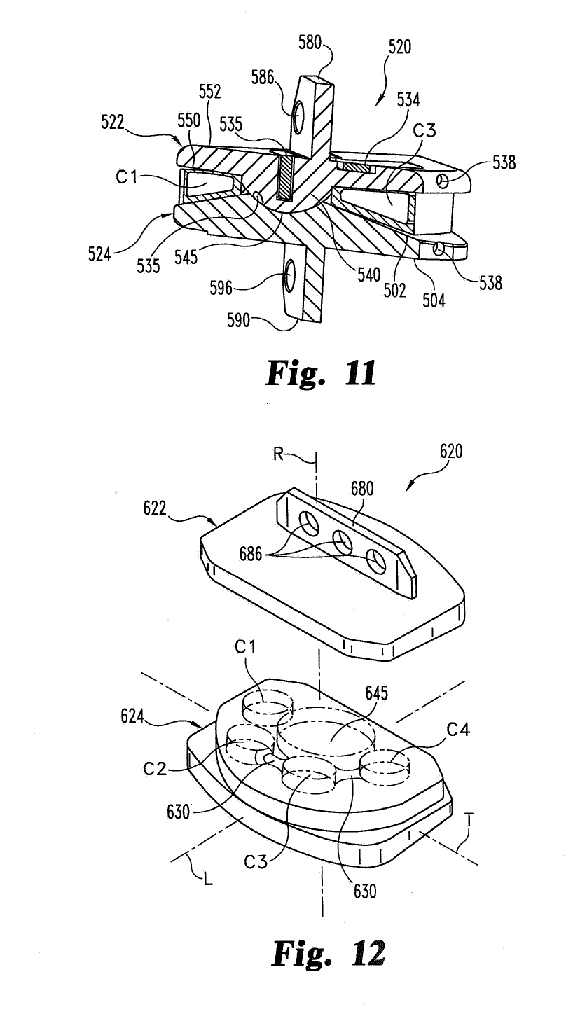

In another embodiment, depicted in FIGS. 10 and 11, device 520 includes both a viscoelastic component 526 and an articulation interface between plate members 522, 524. The articulation interface is provided between an articulation member 540 having a convex shape, which may be configured as a hemispherical-shaped projection from plate member 522 and a concave recess formed into plate member 524 or onto the viscoelastic component 526, or both, in a corresponding position. Projection 540 of plate member 522 is at least partially disposed within the recess or socket 545 of plate member 524. The convex and concave articular surfaces of projection 540 and socket 545 abut one another in such a manner as to provide relative articulating motion between plate members 522, 524. Specifically, plate members 522, 524 are allowed to pivot and rotate relative to one another to maintain or restore motion substantially similar to the normal biomechanical motion provided by a natural intervertebral disc.

In one embodiment, the spherical-shaped surface of projection 540 has a large enough radius of curvature such that the axis about which plate members 522, 524 pivot relative to one another is located at or above the planar surface 550 of plate member 522 (i.e., the center of curvature is located at or above planar surface 550). However, it should be understood that the pivot axis may alternatively be positioned below planar surface 550. It should be understood that other configurations of articulation member 540 are also contemplated, such as, for example, cylindrical, elliptical or other arcuate configurations or possibly non-arcuate configurations. It should also be understood that the remaining portion of plate member 522 may take on planar or non-planar configurations, such as, for example, an angular or conical configuration extending about articulation member 540. In some embodiments, articulation member 540 can be a separate structure from either or both of plate members 522, 524, such as is disclosed in U.S. Pat. Nos. 5,674,296 and 6,019,792, and U.S. Published Application Nos. 2002/0035400 and 2003/0199982, which are hereby incorporated by reference. In other embodiments, including the embodiment depicted in FIGS. 10 and 11, the articulation member 540 can be integral with or otherwise connected to one or both of plate members 522, 524, such as is disclosed in U.S. Pat. Nos. 5,258,031; 6,113,637; and U.S. Published Application No. 2003/0208273, which are hereby incorporated by reference.

In addition to the articulation member 540, a viscoelastic component 526 is interposed between plate members 522, 524. Viscoelastic component 526 can be attached to one or both of plate members 522, 524, and includes at least two fluid-containing chambers disposed in various locations that are in fluid communication with one another through an aperture or channel. In another embodiment, viscoelastic component 526 includes at least three fluid chambers, each of the three fluid chambers fluidly connected to at least one other fluid chamber. In yet another embodiment, viscoelastic component 526 includes at least four fluid chambers, each of the four fluid chambers fluidly connected to at least one other fluid chamber. In yet another embodiment, viscoelastic component 526 includes at least five fluid chambers, each of the five fluid chambers fluidly connected to at least one other fluid chamber. Viscoelastic component 526 provides various functions, including constraining, cushioning, or dampening the relative motion between plate members 522, 524.

In the embodiment depicted in FIGS. 10 and 11, plate member 524 includes a recess 545 in the form of a spherical socket. In some embodiments, recess 545 has a concave shape, and is configured as a spherical-shaped socket. However, it should be understood that other configurations of recess 545 are also contemplated, such as, for example, cylindrical, elliptical or other arcuate configurations or possibly non-arcuate configurations.

Convex articular projection 540 defines a depression or cavity 555 in its convex surface to provide a means for clearing out matter, such as particulate debris, that is disposed between plate members 522, 524. Cavity 555 is operable to capture any such debris, thereby clearing the debris from the interfacing articular surfaces of the device 520 to prevent or at least reduce wear which otherwise might occur if foreign particles and/or built-up wear debris were to remain between the abutting portions of the articular surfaces. Although concave recess 545 is illustrated as having a generally smooth, uninterrupted articular surface, it should be understood that a surface depression or cavity (not shown) may be defined along a portion of the recess 545 to provide a means for clearing out matter, such as particulate debris, that is disposed between plate members 522, 524. In such case, the convex articular surface of projection 540 may alternatively define a generally smooth, uninterrupted articular surface. In other embodiments, each of convex projection 540 and concave recess 545 may define a surface depression to facilitate removal of particulate matter disposed between plate members 522, 524. However, it should be understood that other types of surface depressions are also contemplated. In one embodiment of the invention, the surface depression 555 is configured as a groove. For example, a groove can extend along the convex surface of the spherical-shaped projection 540 in such a manner as to divide projection 540 into two substantially symmetrical portions or, alternatively, can take on other configurations as well. For example, the groove can alternatively be positioned at other locations along projection 540, can be arranged at other angular orientations relative to projection 540, can extend across only a portion of the ball 60, and can take on angular configurations or non-linear configurations, such as, for example, a curvilinear configuration. It should also be understood that any number of grooves may be defined along the periphery of projection 540, such as two or more grooves arranged in a uniform manner or alternatively in a random or semi-random pattern. In still other embodiments, such depression or cavity can be absent from both articular surfaces of projection 540 and recess 545, or recess 545 may include a trough, such as is shown in presently incorporated U.S. Pat. No. 6,113,637 for allowing translational movement between the respective plate members 522, 524.

In the embodiment depicted in FIGS. 10 and 11, the bearing surface 504 of plate member 524 is substantially planar and is oriented at an angle relative to the surface 502, which has a generally frusto-conical shape, to define an outward taper of plate member 524 extending entirely about the concave recess 545. In this manner, relative pivotal motion between plate members 522, 524 is limited by the angle between surface 502 of plate member 524 and surface 550 of plate member 522. In one embodiment, the angle falls within a range of about 10 degrees to about 20 degrees, thereby limiting the overall relative pivotal motion between plate members 522, 524 within a range of just over 20 degrees to just over 40 degrees. In a specific embodiment, the angle is about 16 degrees, thereby limiting the overall pivotal motion between plate members 522, 524 to just over 32 degrees. As will be appreciated by a person of ordinary skill in the art, the angle may take on other values that correspond to the desired amount of relative pivotal movement between plate members 522, 524. It should also be understood that frusto-conical surface 502 may take on other configurations, such as, for example, an angular configuration extending about the concave recess 545. It should also be understood that the surface 502 could alternatively be configured as a planar surface oriented parallel with bearing surface 504, and that surface 550 of plate member 522 could alternatively be configured as a frusto-conical or angled surface tapered at an angle, or that both of the surfaces 550, 502 could alternatively be configured as conical or angled surfaces tapered at a predetermined angle. In an embodiment where both of the surfaces 550, 502 are tapered at a predetermined angle, the angle is preferably about 8 degrees, thereby limiting the overall pivotal motion between plate members 522, 524 to just over 32 degrees.

Pivotal and rotation motion between plate members 522, 524 is also limited by viscoelastic component 526, which is positioned wholly or partially around projection 540 and between plate members 522, 524. Viscoelastic component is in continual contact with the plates 22, 24 and thereby provide a constrained cushion therebetween. The amount of cushioning that is provided can be controlled by factors such as the size of each chamber or the material composition of the viscoelastic component. Also, one or more of the chambers can be filled with a material, such as a gel, that affects flexibility. In yet another embodiment, one or more of the chambers can be filled with a material that changes over time or in response to other conditions, such as those materials discussed above.

In one embodiment (not shown), bearing surface 504 of plate member 424 is substantially planar and is oriented at an angle relative to bearing surface 552 of plate member 522, to define an outward taper extending from an anterior side of device 520 to a posterior side of device 520. In one embodiment, the angle formed between surfaces 504 and 552 falls within a range of 0 degrees to about 10 degrees. However, it should be understood that the angle may take on other values that correspond to the particular lordotic angle or morphology of the portion of the spinal column in which the prosthetic joint 520 is used. However, it should be understood that bearing surfaces 504, 552 may take on alternative configurations, such as, for example, a curved or arcuate configuration that corresponds to the particular contour of the adjacent vertebral endplate against which surfaces 504, 552 abut. It should further be understood that the bearing surfaces 504, 552 may be configured to accommodate spinal abnormalities such as scoliosis. In such case, bearing surfaces 504, 552 may be angled to define a taper extending from one lateral surface to the opposite lateral surface. It should additionally be understood that the bearing surfaces 504, 552 may be roughened and/or may define a number of surface projections to aid in gripping the vertebral endplate and to inhibit migration of the prosthetic joint 520 relative to the adjacent vertebra.

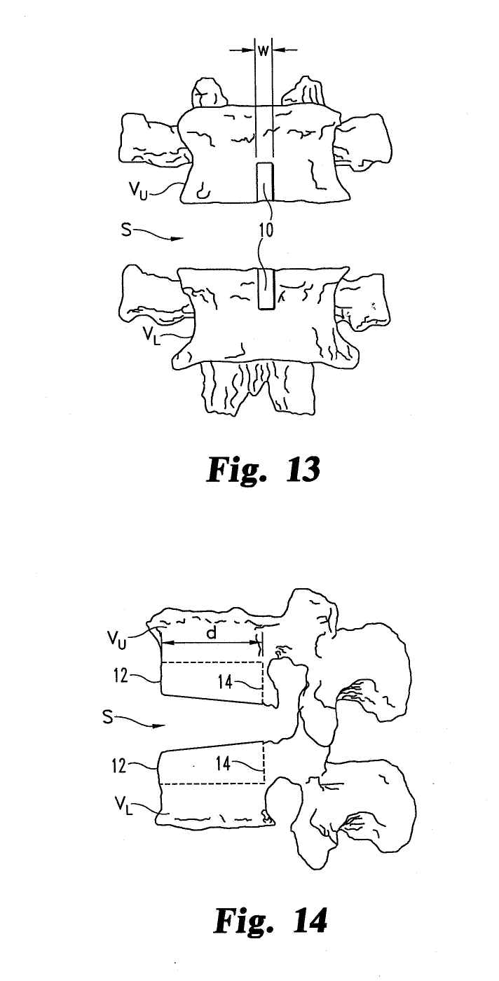

For purposes of discussing the placement of implant devices in accordance with the present application into a vertebral location, reference will be made to the implant embodiment 20 depicted in FIGS. 2-4. As illustrated in FIGS. 13 and 14, removal of the diseased or degenerated disc D results in the formation of an intervertebral space S between the upper and lower vertebrae V_{U}, V_{L}. To accommodate insertion of prosthetic joint 20, within the intervertebral space S, preparation of the upper and lower vertebrae V_{U}, V_{L} is required to accept the prosthetic joint therebetween. Specifically, elongate openings or slots 10 are formed along the vertebral endplates of the upper and lower vertebrae V_{U}, V_{L} at a predetermined width w and to a predetermined depth d. In one embodiment, the elongate slots 10 are rectangular-shaped and extend from an anterior side 12 of the vertebrae V_{U}, V_{L} toward a posterior side 14 of the vertebrae V_{U}, V_{L}. In a specific embodiment, slots 10 are formed by chiseling or curetting. However, other methods of forming slots 10 are also contemplated as would occur to one of ordinary skill in the art, such as, for example, by drilling or reaming. In a preferred embodiment, the width w of slots 10 is equal to or somewhat less than the corresponding width of keels 80, 90 of plate members 22, 24. Additionally, the depth d of slots 10 is preferably approximately equal to or slightly greater than the length of keels 80, 90.

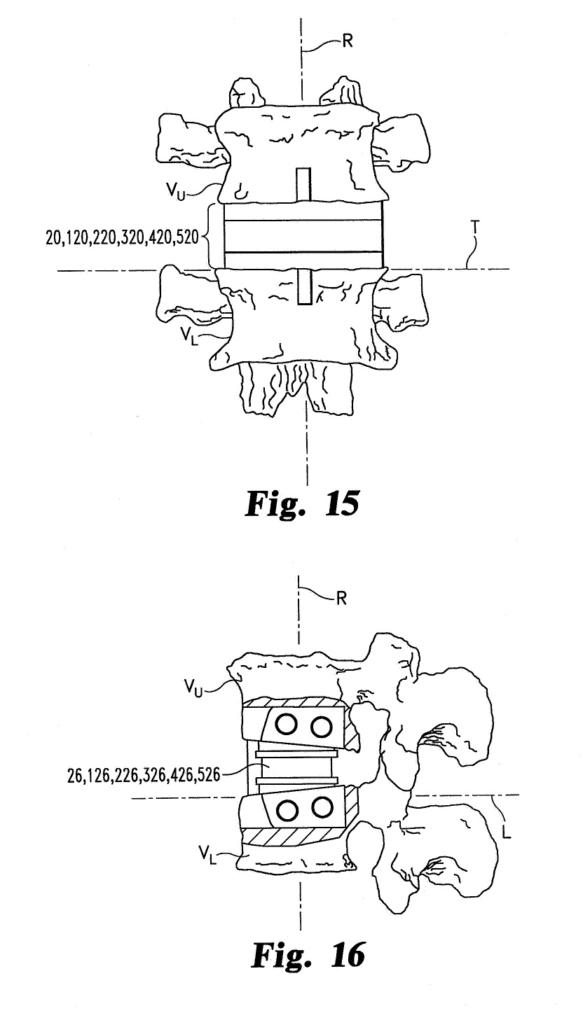

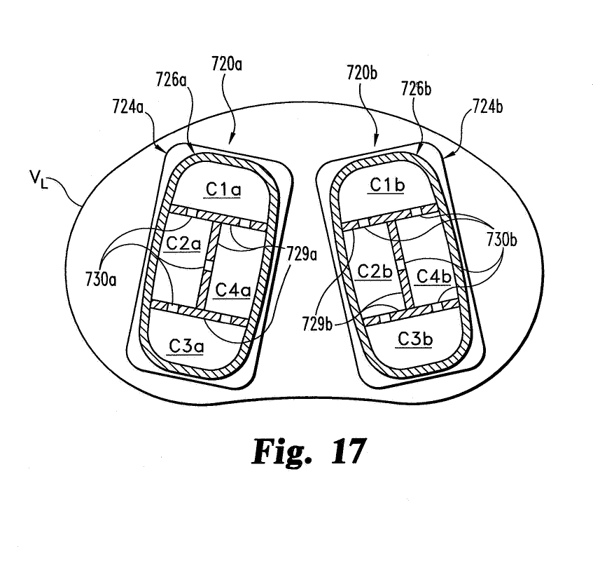

Referring to FIGS. 15 and 16, following preparation of the intervertebral space S, plate members 22, 24 are inserted between the upper and lower vertebrae V_{U}, V_{L}. First, plate members 22, 24 are placed in a predetermined relationship with respect to one another, preferably by an insertion instrument (not shown) or an equivalent tool that is adapted to engage channels formed along a length of plate members 22, 24 or recessed formed into the anterior-facing side of plate members 22, 24. The insertion instrument (not shown) holds plate members 22, 24 in a predetermined spatial relationship and at a predetermined orientation with respect to one another. The prosthetic joint 20 is inserted between the upper and lower vertebrae V_{U}, V_{L} in a direction generally along the longitudinal axis L, with keels 80, 90 of plate members 22, 24 being axially displaced along the slots 10. Notably, since keels 80, 90 are axially displaced through the preformed slots 10, distraction of upper and lower vertebrae V_{U}, V_{L} to accommodate insertion of the prosthetic joint 20 is minimized, if not eliminated entirely.

As discussed above, keels 80, 90 can be tapered or wedge-shaped to facilitate insertion within slots 10. Since the width w of slots 10 is equal to or somewhat less than the corresponding width of keels 80, 90, keels 80, 90 are effectively wedged within slots 10. The depth d of slots 10 formed in the upper and lower vertebrae V_{U}, V_{L} correspondingly controls the positioning of prosthetic joint 20 within intervertebral space S. Specifically, proper positioning of prosthetic joint 20 is accomplished when the insertion ends 80a, 90a of keels 80, 90 bottom out against the end surfaces of slots 10. Controlling the insertion depth of prosthetic joint 20 results in more precise positioning to avoid over-insertion or under-insertion of prosthetic joint 20. As discussed above, the angular positioning of plate members 22, 24 relative to one another is dictated by the geometry of the upper and lower vertebrae V_{U}, V_{L} and the particular location within the spinal column. As should be apparent, the distance between the bearing surfaces 54, 104 of plate members 22, 24 should be approximately equal to the height of the removed disc D, and the angular disposition of bearing surfaces 54, 104 is dictated by the particular curvature or lordosis of the spinal column.

Once prosthetic joint 20 is inserted within the intervertebral space S, plate members 22, 24 are initially secured to the upper and lower vertebrae V_{U}, V_{L} via the disposition of keels 80, 90 within slots 10 formed in vertebrae V_{U}, V_{L} and by the compression forces exerted upon bearing surfaces 54, 104 of plate members 22, 24 by the adjacent vertebral endplates. Keels 80, 90 thus serve to resist migration or displacement of prosthetic joint 20 relative to adjacent vertebrae V_{U}, V_{L}. Subsequent to the implantation of prosthetic joint 20, plate members 22, 24 are further secured to the upper and lower vertebrae V_{U}, V_{L} via bone growth through the openings 86, 96 in keels 80, 90 and/or by bone on-growth onto bearing surfaces 54, 104 of plate members 22, 24 that are in direct contact with vertebral bone. The bone through-growth and bone on-growth provide further resistance to the migration or displacement of prosthetic joint 20 and prevent expulsion of prosthetic joint 20 from intervertebral space S. It should be understood that other means of engaging prosthetic joint 20 to upper and lower vertebrae V_{U}, V_{L} are also contemplated, such as, for example, by bone screws, staples, an adhesive, or by other methods of engagement as would occur to one of ordinary skill in the art.

In use, plate members 22, 24 cooperate with one another to provide a joint that permits relative pivotal and rotational movement therebetween, which correspondingly permits relative pivotal and rotational movement between the upper and lower vertebrae V_{U}, V_{L}. As a result, substantially normal biomechanical motion is restored to the portion of the spinal column being treated. Although the devices and methods described in the present application are particularly applicable to the lumbar region of the spine, it should nevertheless be understood that the present application is also applicable to other portions of the spine, including the cervical or thoracic regions of the spine.

In the embodiment illustrated in FIGS. 13-16, prosthetic joint 20 is implanted in the intervertebral space S via an anterior approach. However, it should be understood that the slots 10 may alternatively extend from the posterior side of vertebrae V_{U}, V_{L} toward the anterior side at a depth d, and the prosthetic joint 20 may alternatively be implanted in the intervertebral space S via a posterior approach. It should also understood that the slots 10 may alternatively extend from a first lateral side of vertebrae V_{U}, V_{L} toward the opposite lateral side of the vertebrae at a depth d, and the prosthetic joint 20 may alternatively be implanted in the intervertebral space S via a lateral approach. In still other alternative manners of practicing the invention, the slots are positioned at suitable locations for insertion of an implant device from a posterolateral approach. As will be appreciated by a person of ordinary skill in the art, keels 80, 90 can be positioned at a wide variety of angles relative to plate members 22, 24 so that the insertion angle of keels 80, 90 corresponds to alternate slot positions, thereby accommodating insertion of prosthetic joint 20 from a wide variety of approaches. For example, with reference to FIG. 12, implant 620 has many features in common with implant 520; however, implant 620 is configured for insertion into an intervertebral space S via a lateral approach. Specifically, plate 622 of implant 620 includes keel 680 that extends between lateral edges of plate 622 rather than between anterior and posterior facing edges.

In addition to the embodiments described above, the present application also contemplates positioning multiple implant devices in the same interbody space. In one embodiment, an implant system includes two implant devices including viscoelastic components as described herein in the same interbody space to provide a bilateral implant system. In such a system, represented schematically in FIG. 17, devices 720a, 720b are positioned bilaterally in an interbody space between vertebrae V_{U}, V_{L}. In the embodiment show, device 720a includes viscoelastic component 726a that defines four chambers C1a, C2a, C3a, C4a within walls 729a. Each of chambers C1a, C2a, C3a, C4a is fluidly connected to one or more adjacent chambers through channels or apertures 730a. Viscoelastic component 726a is positioned between plate 724a and plate 722a (not shown). Device 720b includes viscoelastic component 726b that defines four chambers C1b, C2b, C3b, C4b within walls 729b. Each of chambers C1b, C2b, C3b, C4b is fluidly connected to one or more adjacent chambers through channels or apertures 730b. Viscoelastic component 726b is positioned between plate 724b and plate 722b (not shown). Devices 720a and 720b can include a wide variety of alternative features similar to those described above in connection with embodiments 20, 120, 220, 320, 420.

In another embodiment (not shown), the system includes at least three implant devices positioned in the same interbody space, and at least two of the at least three implant devices includes a viscoelastic component as described herein. In one embodiment, all of the at least three implant devices includes a first plate member for engaging with a first vertebral body; a second plate member for engaging with a second vertebral body; and a viscoelastic component between the two plate members. In another embodiment, at least one of the devices does not include a viscoelastic component. For example, one device of the system (not shown) can be an articulating implant of a type known in the prior art, with at least two implant devices positioned in the interbody space at locations spaced apart from the articulating device, each of which includes a viscoelastic component positioned between two plate members.

While the invention has been illustrated and described in detail in the drawings and foregoing description, the same is to be considered as illustrative and not restrictive in character, it being understood that all changes and modifications that come within the spirit of the invention are desired to be protected. Any alterations and further modifications in the described embodiments, and any further applications of the principles of the invention as described herein are contemplated as would normally occur to one skilled in the art to which the invention relates. Moreover, individual features of separately described embodiments can be combined to form additional embodiments. In addition, reference numerals are repeated throughout many of the embodiments. Such repetition does not indicate that features of some embodiments must be or should be used with other embodiments. Instead, a wide assortment of different embodiments with one or more features from various drawings and discussions is intended.

What is claimed is:

1. A motion-preserving implant device comprising:
a first plate member for engaging with a first vertebral body;
a second plate member for engaging with a second vertebral body;
a viscoelastic component between the two plate members, said viscoelastic component defining at least two fluid chambers having elastic side walls and at least one channel fluidly connecting said at least two chambers; and
a fluid contained within said chambers.

2. The device of claim 1 wherein the viscoelastic component is configured to constrain the relative motion between the two plate members.

3. The device of claim 1 wherein the viscoelastic component is configured to provide pivotal and rotational movement between the two vertebral bodies.

4. The device of claim 1 wherein the viscoelastic component is attached to at least one of said first and second plate members via an attachment mechanism.

5. The device of claim 1 wherein said channel comprises a valve.

6. The device of claim 1 wherein said fluid is a liquid.

7. The device of claim 1 wherein the fluid comprises a liquid selected from the group consisting of water, saline, polyethylene glycol, glycerol, plasma extender and hydrocarbon solvents.

8. The device of claim 1 wherein the fluid has a viscosity of from about 1 to about 250,000 Centipoise.

9. The device of claim 1 wherein at least one of said chambers is filled with a gel.

10. The device of claim 1, further comprising a central load bearing chamber defined by durable flexible walls, wherein said central load bearing chamber is not in fluid communication with said at least two fluid chambers.

11. The device of claim 1 wherein said fluid comprises a material that changes properties in response to an external stimulus.

12. The device of claim 1 wherein said fluid is a magnetic rheological fluid.

13. The device of claim 12, further comprising a magnetic field source.

14. The device of claim 12 wherein said magnetic field source comprises one or more permanent magnets and one or more electromagnets.

15. The device of claim 14, further comprising a power source and a microelectronic controller for said electromagnet and a compartment for housing said power source and microelectronic controller.

16. An intervertebral prosthetic device, comprising:
a first component adapted to engage a first vertebra and including a first articular surface;
a second component adapted to engage a second vertebra and including a second articular surface that cooperates with said first articular surface at an articulating interface to permit articulating motion between said first and second components;
a viscoelastic component positioned about said articulating interface, said viscoelastic component defining at least two fluid chambers having elastic side walls and at least one channel fluidly connecting said at least two chambers; and
a fluid contained within said chambers.

17. The device of claim 16 wherein the articulating interface is configured to provide rotational and translational movement between the two vertebral bodies.

18. The device of claim 16, wherein one of said first and second articular surfaces comprises a convex surface, another of said first and second articular surfaces comprises a concave surface, at least a portion of said convex surface abutting at least a portion of said concave surface to permit said articulating motion.

19. The device of claim 16, wherein said convex and concave surfaces are substantially spherical-shaped.

20. An intervertebral prosthetic joint system, comprising two implant devices configured for bilateral placement in an interbody disc space, each of said devices comprising a first plate member for engaging with a first vertebral body; a second plate member for engaging with a second vertebral body; and a viscoelastic component between the two plate members, said viscoelastic component including at least two fluid chambers, at least one channel fluidly connecting said at least two chambers, and a fluid contained within said chambers.
